(19) United States
(12) Patent Application Publication
(10) Pub. No.: US 2011/0207230 A1
(43) Pub. Date: Aug. 25, 2011

(54) EVALUATION OF 24S-HYDROXYCHOLESTEROL IN HAIR FOR METABOLIC BIOMARKER OF ALZHEIMER'S DISEASE

(75) Inventors: Bong Chul CHUNG, Namyangju Si (KR); Man Ho Choi, Seoul (KR); Kyung Mi Kim, Seoul (KR)

(73) Assignee: KOREA INSTITUTE OF SCIENCE AND TECHNOLOGY, Seoul (KR)

(21) Appl. No.: 12/712,574

(22) Filed: Feb. 25, 2010

(51) Int. Cl. G01N 33/92

(52) U.S. Cl. 436/71

(57) ABSTRACT

Disclosed is a method for detection of 24S-hydroxycholesterol in hair. More specifically, a trace amount of hair is hydrolyzed under basic condition and metabolites including 24S-hydroxycholesterol are purified and extracted by solid-phase extraction (SPE). Then, after derivatization of the resulting extract with trimethylsilyl (TMS), 24S-hydroxycholesterol is detected by gas chromatography-mass spectrometry (GC-MS).

### BACKGROUND OF THE INVENTION

1. Field of the Invention

The present invention relates to a method for quantification of 24S-hydroxycholesterol in hair and evaluation of its change along with progression of Alzheimer's disease

2. Description of Related Art

24S-hydroxycholesterol is a main brain-generated cholesterol metabolite which is associated with neurodegeneration. It is generated from cholesterol by cholesterol 24S-hydroxylase (CYP46). It has been experimentally demonstrated that patients with Alzheimer's disease show an increased level of 24S-hydroxycholesterol in brain tissues, cerebrospinal fluid (CSF) or blood as compared to healthy people [J. Lipid Res., 2000, 41:195-8 & 2004, 45:186-193; Pharmacopsychiatry, 2003, 36:S102-S106]. However, diagnosis of Alzheimer's disease via monitoring the increased level of 24S-hydroxycholesterol in brain tissue and CSF is too much invasive as clinical sampling for early diagnosis, as well as its blood levels does not give significant differences between patients and healthy people. Therefore, there is a need an alternative sampling method to evaluate the level of 24S-hydroxycholesterol for early diagnosis of Alzheimer's disease [Curr. Alz. Res., 2005, 2:47-64].

Recently, analysis of metabolites in hair has been utilized in clinical diagnosis [J. Invest. Dermatol., 2001, 116:57-61; Clin. Chem., 2001, 47:143-144]. Hair has advantages over urine and blood in that the processes of collecting and storage are rather easy. In addition, analysis of hair has been drawing much attention as a supplement for blood and urine tests which do not reflect past physical conditions of patients. Accordingly, measurement of 24S-hydroxycholesterol level and analysis of metabolite distribution in hair may be served as an experimental tool for assaying physiological change caused by Alzheimer's disease.

### SUMMARY OF THE INVENTION

The inventors of the present invention hydrolyzed a trace amount of hair corresponding to one strand under basic condition, extracted all metabolites including 24S-hydroxycholesterol by solid-phase extraction (SPE), measured 24S-hydroxycholesterol level and analyzed distribution of all the metabolites in hair via trimethylsilyl (TMS) derivatization and gas chromatography-mass spectrometry (GC-MS), and represented the distribution pattern.

Accordingly, an object of the present invention is to provide a method for detection of 24S-hydroxycholesterol level in a trace amount of hair sample and diagnosis of Alzheimer's disease by monitoring of change in metabolites by representing the distribution pattern of hair metabolites analyzed.

The present invention provides a method for detection of 24S-hydroxycholesterol, including:

hydrolyzing hair under basic condition and extracting metabolites including 24S-hydroxycholesterol using a copolymer adsorbent having lipophilic and hydrophilic moieties; and

derivatizing the resulting extract with trimethylsilyl (TMS) and measuring the level of 24S-hydroxycholesterol in hair by gas chromatography-mass spectrometry (GC-MS).

The present invention further provides a method for evaluation of a metabolic biomarker of Alzheimer's disease, including representing a distribution pattern of metabolites including 24S-hydroxycholesterol existing in hair, which is obtained using the afordescribed method for detection, graphically by means of a statistical method, and comparing the result between a patient with Alzheimer's disease and a healthy person.

The present invention provides a method for extraction and analysis of metabolites including 24S-hydroxycholesterol from a trace amount of hair. This allows the detection of 24S-hydroxycholesterol level in hair, as well as distribution pattern of other metabolites. Accordingly, the present invention may be utilized as a tool for diagnosis of Alzheimer's disease.

### BRIEF DESCRIPTION OF THE DRAWINGS

The above and other objects, features and advantages of the present invention will become apparent from the following description of preferred embodiments given in conjunction with the accompanying drawing, in which:

FIG. 1 shows a result of analyzing 24S-hydroxycholesterol in a strand of hair taken from 24 female patients (58-85 years old, 68.2±7.8 years; mean±SD) with Alzheimer's disease and 23 healthy women of matched ages (56-75 years old, 61.8±7.0 years);

FIG. 2 graphically shows a possibility of distinguishing a patient with Alzheimer's disease from a healthy person; and

FIG. 3 shows a statistical clustering of a distribution pattern of hair metabolites including 24S-hydroxycholesterol based on partial least squares-discriminant analysis (PLS-DA).

### DESCRIPTION OF SPECIFIC EMBODIMENTS

The advantages, features and aspects of the invention will become apparent from the following description of the embodiments with reference to the accompanying drawings, which is set forth hereinafter.

According to the present invention, a trace amount of hair is hydrolyzed under basic condition and metabolites including 24S-hydroxycholesterol are extracted by solid-phase extraction (SPE). Then, after trimethylsilyl (TMS) derivatization, 24S-hydroxycholesterol is quantified and a distribution pattern of the all metabolites extracted is evaluated by gas chromatography-mass spectrometry (GC-MS).

First, hair is hydrolyzed under basic condition. Preferably, the amount of hair used is 1 to 20 mg. If the amount is less than 1 mg, the quantity of the metabolites including 24S-hydroxycholesterol may be smaller than the quantification limit. Meanwhile, if the amount is more than 20 mg, there may be an excess quantity of cholesterol or other substances in addition to the inconvenience of pulling out a lot of hair.

Then, hair is hydrolyzed at 60° C. for 1 hour and metabolites including 24S-hydroxycholesterol are extracted using a copolymer adsorbent having lipophilic and hydrophilic moieties. In an embodiment, an Oasis HLB cartridge providing high recovery of biomolecules and giving reproducible analysis result may be used (Rapid Commun. Mass Spectrom., 2002, 16:2221-2228). The metabolites include all metabolites existing in hair, including, for example, steroids, fatty acids, organic acids and amines.

Then, hydrolysate adsorbed on the SPE adsorbent is eluted with methanol, and the eluate is evaporated and dried to obtain an extract. Then, the obtained extract is dried and subjected to GC-MS analysis after TMS derivatization.

The peak area of 24S-hydroxycholesterol in hair obtained from GC-MS is compared with that of reference standard to determine the concentration of 24S-hydroxycholesterol. Based on comparison of 24S-hydroxycholesterol level in hair between patients with Alzheimer's disease and healthy people, a higher level than the average of healthy people may be diagnosed as Alzheimer's disease.

In addition, a distribution pattern of the metabolites including 24S-hydroxycholesterol existing in hair determined as described above may be prepared into a metabolite array for evaluation of a metabolic biomarker for Alzheimer's disease.

### EXAMPLES

The examples and experiments will now be described. The following examples are for illustrative purposes only and are not intended to limit the scope of the present invention.

### Example 1

### Detection of Metabolites Including 24S-Hydroxycholesterol

1) Hydrolysis

Hair was washed sufficiently with distilled water and isopropyl alcohol to remove contaminants on the surface and, after drying, was cut to a size of 1 to 2 mm. After adding internal standard (d6-cholesterol, 1 μg/mL, 50 μL) and hair (1 mg) to a test tube, followed by addition of 6 M KOH (1 mL), ethanol (1 mL) was further added and hydrolysis was carried out at 60° C. for 1 hour [Biomed. Chromatogr., 2006, 20:999-1003].

2) Solid-Phase Extraction (SPE)

Oasis HLB cartridge [60 mg, Waters, Co., Milford, Mass., USA] was used for SPE. Methanol and distilled water (1 mL each) were flown 2 times, and then the hydrolysate of 1) was loaded. After washing with distilled water (1 mL) to remove polar impurities, water was sufficiently removed under reduced pressure. Then, methanol (1.5 mL) was flown 2 times to elute the hydrolysate of 1) adsorbed on the cartridge. The resulting eluate was collected in a fresh test tube. The eluate was evaporated using a rotary evaporator at 40° C. and then dried sufficiently in a vacuum desiccator using P205/KOH for at least 30 minutes.

3) Gas Chromatography-Mass Spectrometry (GC-MS)

An N-methyl-N-trifluorotrimethylsilylacetamide (MSTFA)/ammonium iodide (NH₄I)/dithioerythritol mixture solution (1000:4:5, v/w/w, 50 μL) was added to the dried residue and derivatization was performed at 60° C. for 20 minutes.

Selected-ion monitoring (SIM) analysis, which can selectively detect specific ions of 24S-hydroxycholesterol, was carried out using Agilent 5975 Mass Selective Detector and Agilent 6890 Series Gas Chromatograph. Three specific ions (m/z: 546, 503, 413) were analyzed. For analysis of metabolites including 24S-hydroxycholesterol, the Polaris GC-MS system (ThermoFinnigan) was used in a scan mode of masses 50 to 650. Agilent Ultra-1 fused-silica capillary column (25 m long with 0.2 mm of inner diameter) was used for the analysis of metabolites including 24S-hydroxycholesterol. Film thickness was 0.33 μm and flow rate of the carrier gas helium was 0.9 mL/min. Inlet temperature was set at 260° C. and the sample was introduced in 10:1 split mode. Temperature condition for analysis was as follows. The initial temperature of the oven was set at 240° C. After maintaining the temperature for 0.2 minute, it was increased to 290° C. at a rate of 40° C./min and kept for 9 minutes. Finally, after raising the temperature to 320° C. at a rate of 30° C./min, it was maintained for 2 minutes. Detector temperature was set at 280° C. and ion source temperature was set at 230° C. Ionization was performed by electron impact ionization (EI) with an electron energy of 70 eV. In order to determine the concentration of 24S-hydroxycholesterol, the area ratio of the m/z 413 peak obtained from SIM was compared with that of the standard material. For analysis of other metabolites, the scan mode enabling analysis of all the compounds with masses between 50 and 650 was used.

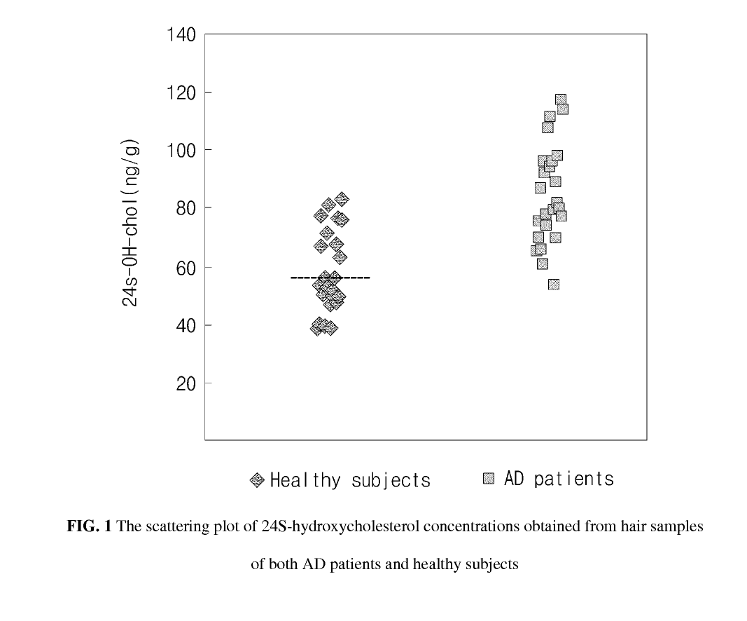

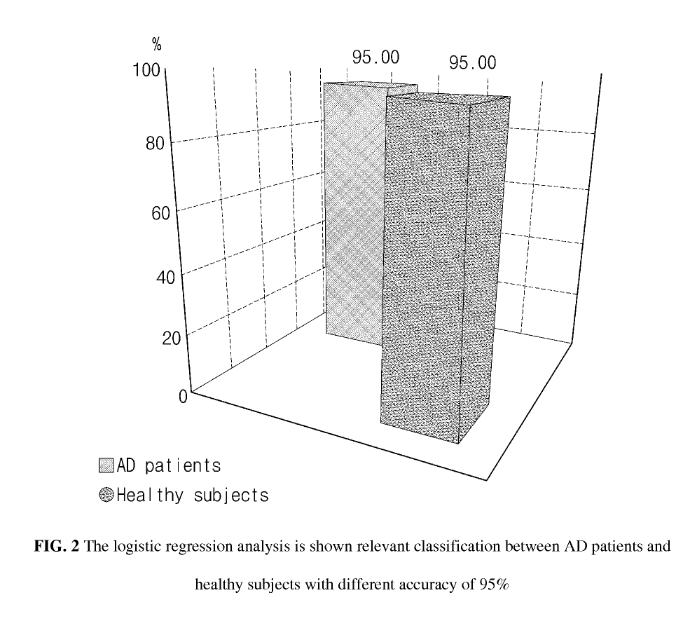

The level of 24S-hydroxycholesterol in hair taken from 24 female patients with Alzheimer's disease and 23 healthy subjects was compared. The result is shown in FIG. 1. The mean of the 24S-hydroxycholesterol concentration in the patients with Alzheimer's disease and the healthy people was 83.9 ng/g and 58.4 ng/g, respectively (p<0.001). The possibility of distinguishing them with statistical significance was 95% (see FIG. 2).

### Example 2

### Analysis of Distribution Pattern of Metabolites in Hair

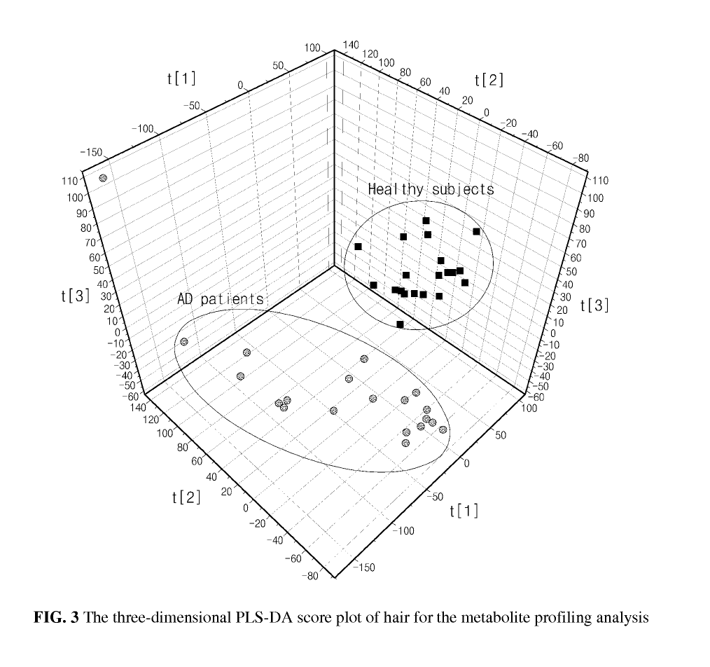

A distribution pattern of all the metabolites existing in a strand of hair taken from the patients with Alzheimer's disease and the healthy people detected in Example 1 was determined by partial least squares-discriminant analysis (PLS-DA) [Anal. Chem., 2007, 79:6102-6110]. The result is shown in FIG. 3. As shown in FIG. 3, the distribution pattern of the metabolites in hair could be distinguished depending on the presence or absence of Alzheimer's disease. This suggests that distinction between patients with Alzheimer's disease from healthy people is possible not only with the comparison of 24S-hydroxycholesterol level but also with the metabolite array.

While the present invention has been described with respect to the specific embodiments, it will be apparent to those skilled in the art that various changes and modifications may be made without departing from the spirit and scope of the invention as defined in the following claims.

1. A method for screening for Alzheimer's disease by detecting the presence of 24S-hydroxycholesterol in a sample of hair from a human suspected to have Alzheimer's disease, comprising:
hydrolyzing hair under basic condition and extracting metabolites including 24S-hydroxycholesterol using a copolymer adsorbent having lipophilic and hydrophilic moieties; and
derivatizing the resulting extract with trimethylsilyl and measuring the hair concentration of 24S-hydroxycholesterol by gas chromatography-mass spectrometry (GC-MS),
wherein, the concentration of 24S-hydroxycholesterol which is statistically significantly greater than an average concentration of 24S-hydroxycholesterol in healthy people indicates a preliminary diagnosis of Alzheimer's disease.

2. The method for screening according to claim 1, wherein the amount of the hair is 1 to 20 mg.

3. The method for screening according to claim 1, further comprising, after said measuring, comparing the 24S-hydroxycholesterol concentrations in hair between a patient with Alzheimer's disease and a healthy person, wherein the average concentration of 24S-hydroxycholesterol in healthy people is about 58.4 ng/g.

4. A method for evaluation of a metabolic biomarker of Alzheimer's disease, comprising representing a distribution pattern of metabolites including 24S-hydroxycholesterol existing in hair, which is obtained using a method for screening for Alzheimer's disease by detecting the presence of 24S-hydroxycholesterol in a sample of hair from a human suspected to have Alzheimer's disease, graphically as a metabolite array by a statistical method, wherein the method for screening 24S-hydroxycholesterol comprises hydrolyzing the sample of hair under basic condition and extracting metabolites including 24S-hydroxycholesterol using a copolymer adsorbent having lipophilic and hydrophilic moieties; and derivatizing the resulting extract with trimethylsilyl and measuring the hair concentration of 24S-hydroxycholesterol and the metabolites by gas chromatography-mass spectrometry (GC-MS), wherein, the concentration of 24S-hydroxycholesterol which is statistically significantly greater than an average concentration of 24S-hydroxycholesterol in healthy people indicates a preliminary diagnosis of Alzheimer's disease.

5. The method for evaluation according to claim 4, wherein the statistical method is partial least squares-discriminant analysis (PLS-DA).

6. The method for evaluation according to claim 4, wherein the amount of the hair is 1 to 20 mg.

7. The method for evaluation according to claim 4, wherein the average concentration of 24S-hydroxycholesterol in healthy people is about 58.4 ng/g.

8. A method for screening for Alzheimer's disease by detecting the presence of 24S-hydroxycholesterol in a sample of hair from a human suspected to have Alzheimer's disease, comprising:
hydrolyzing hair under basic condition and extracting metabolites including 24S-hydroxycholesterol using a copolymer adsorbent having lipophilic and hydrophilic moieties;
derivatizing the resulting extract with trimethylsilyl and measuring the hair concentration of 24S-hydroxycholesterol by gas chromatography-mass spectrometry (GC-MS); and
after said measuring, comparing the 24S-hydroxycholesterol concentrations in hair between a patient with Alzheimer's disease and a healthy person, wherein, the concentration of 24S-hydroxycholesterol which is statistically significantly greater than an average concentration of 24S-hydroxycholesterol in healthy people indicates a preliminary diagnosis of Alzheimer's disease, wherein further the 24S-hydroxycholesterol concentrations in the sample of hair from the human suspected to have Alzheimer's disease is about 43.67% more than the average concentration in a sample of hair from a healthy person.

9. The method for screening according to claim 8, wherein the amount of the hair is 1 to 20 mg.
